(19) United States
(12) Patent Application Publication
(10) Pub. No.: US 2016/0150976 A1
(43) Pub. Date: Jun. 2, 2016

(54) HIGH-RESOLUTION THERMAL IMAGING SYSTEM, APPARATUS, METHOD AND COMPUTER ACCESSIBLE MEDIUM

(71) Applicant: THE GENERAL HOSPITAL CORPORATION, Boston, MA (US)

(72) Inventors: Qianqian Fang, North Reading, MA (US); Bernhard Zimmermann, Cambridge, MA (US); Linda T. Wang, Newton, MA (US); Patricia Hibberd, Lexington, MA (US); Robert Cleveland, Newton Highlands, MA (US); Jim Lewis, Brookline, MA (US)

(73) Assignee: The General Hospital Corporation, Boston, MA (US)

(21) Appl. No.: 14/900,485

(22) PCT Filed: Jun. 25, 2014
(86) PCT No.: PCT/US2014/044125

§ 371 Date: Dec. 21, 2015

Related U.S. Application Data
(60) Provisional application No. 61/839,137, filed on Jun. 25, 2013.

(51) Int. Cl. A61B 5/01; A61B 5/00

(52) U.S. Cl. CPC A61B 5/015; A61B 5/7282; A61B 5/0077; A61B 5/7246; A61B 5/0035; A61B 5/7275; A61B 5/6898; A61B 2562/0219; A61B 2576/00

(57) ABSTRACT

Exemplary of the present disclosure can include an apparatus for generating thermal imaging information of a biological structure(s), including detector arrangement(s) which can be configured to receive and detect light radiation(s), a determination arrangement(s) which can be configured to determine a position or an orientation of the detector arrangement(s) with respect to the biological structure(s) to generate data, and a computer arrangement configured to generate the thermal imaging information based on the radiation and the data when the detector arrangement(s) is moved. The thermal imaging information can include a 2-dimension temperature map(s) of a portion(s) of the biological structure(s). The thermal imaging information can include a 3-dimension temperature map(s) of a portion(s)

### CROSS-REFERENCE TO RELATED APPLICATIONS

This application relates to and claims priority from U.S. Patent Application No. 61/839,137, filed on Jun. 25, 2013, the entire disclosure of which is incorporated herein by reference.

### FIELD OF THE DISCLOSURE

The present disclosure relates generally to a thermal imaging system, and more specifically, to exemplary embodiments of an exemplary thermal imaging system, device, apparatus, method and computer-accessible medium for diagnosing a disease or condition.

### BACKGROUND INFORMATION

The slow progress towards the United Nations Millennium Development Goal #4—to reduce childhood mortality in developing countries by two thirds by 2015—has been partly related to the lack of progress in early recognition, diagnosis and treatment of childhood pneumonia and neonatal pneumonia. Severe pneumonia can be one of the most common reasons that children under age 5 die (see e.g., Reference 11), but can often be elusive and difficult to diagnose in young infants and children based on history and physical examination alone. (See e.g., References 2, 5, 7 and 9). In well-equipped health care facilities, chest x-rays can be used to aid in the diagnosis of severe pneumonia, and to guide critical treatment decisions (e.g., whether a patient needs parenteral antibiotics or not, hospitalization or not, use of oxygen or not). However, in low and middle-income countries (“LMIC”s), children are typically taken to first-level health clinics where chest x-rays may not be available, and the diagnosis of severe pneumonia can often be missed.

Distinguishing pneumonia from bronchiolitis and other viral respiratory infections can be especially difficult in infants and young children. Treatment guidelines from the American Academy of Pediatrics (“AAP”), the American College of Chest Physicians (“ACCP”), and the Centers for Disease Control and Prevention (“CDC”) all recommend not treating viral illnesses with antibiotics. However, for any patient with cough and fever, it can be important to be sure that the patient does not have pneumonia, because pneumonia can be more likely due to bacteria and does need antibiotic treatment. By detecting temperature patterns, case reports in technical and scientific articles have demonstrated thermal imaging has a strong diagnostic potential for detecting lobar consolidation or asymmetric infiltrates consistent with the diagnosis of pneumonia. (See e.g., References 6 and 10).

Thermal imaging capability can provide frontline healthcare workers with a portable and durable technology to make quick, appropriate decisions to distinguish bacterial pneumonia (e.g., a lower respiratory tract disease) from other respiratory infections, such as acute bronchiolitis (e.g., a lower respiratory tract disease) or acute bronchitis (e.g., an upper respiratory tract disease), which are usually caused by viruses over 90% of the time. With a point-of-care thermal imaging device, a healthcare worker can more appropriately guide the timely use of antibiotics for those who have pneumonia. This can be important for resource-poor settings, where medicines and resources can be extremely limited. The scant doses of antibiotics, single oxygen canisters or resources to transfer a patient to the district hospital can be given only to those with severe pneumonia. In addition, the information provided by a thermal scanner can assay in the evaluation of patients of any age, providing critical information to guide healthcare workers to better triage the patient pool to improve health outcomes.

Body temperature can be one of the vital signs routinely measured by healthcare providers and veterinarians to detect or monitor medical problems. Body temperature can vary by gender, time of day, recent activity, and other factors, but normally can range from about 36.5° C. to 37.2° C. Temperatures higher and/or lower than this range can indicate the presence of infection, inflammation, fever and other processes.

Thermometry has been extensively used throughout the history of medicine. Traditional thermometers need direct contact and typically have a slow response. Infrared thermometry can be a technique measuring the thermal infrared radiation from a heated body, and thus does not need direct contact. Since its discovery in the early 1900's, infrared thermometry has been increasingly used for non-contact temperature assessment in medicine, civil engineering and science, from point measurements to temperature maps in the form of a thermal image. A thermal camera can be attractive because it can scan a large area in real-time. It has been widely used in airports to rapidly identify fever and infectious diseases in a high-traffic area. Because thermal infrared radiation, with wavelength ranging from about 9,000 nm to about 14,000 nm, can be invisible to the naked eyes of a human, a thermal camera can also be used for the purpose of night-vision.

Optical sensors, such as CCD and CMOS, have a limited sensitivity in the near-infrared (“NIR”) range and thus can be adapted to obtain thermal images. However, a suitable temperature range of these sensors can be relatively high, typically above 280° C. Specialized thermal imaging sensors, such as Focal Plane Arrays (“FPA”), can directly measure the thermal infrared radiations radiated from a wide range of temperatures. FPA-based thermal cameras are generally more affordable compared to the CCD/CMOS based thermal cameras; however, its resolution can be quite limited, only in the range of a few hundred pixels in each axis of the image. Because an FPA sensor can be more complicated to fabricate, a high-resolution FPA sensor can be very high in cost.

With over 6 billion mobile phones being actively used worldwide, there is one for every person over the age 10. Technological advances have contributed to the development of the smart phone, which can be a cellular phone that has built-in applications and internet access. Despite an uneven smartphone adoption rate, it is estimated that by 2015, over 2 billion smart phones will be in use globally. Africa's one billion people have 750 million phones, and by the year 2016 nearly 30% will be smart phones. (See e.g., Reference 3). A smart phone can combine the capabilities of connectivity, imaging and computing into a single, portable and low-cost platform, and has enormous potential for improving healthcare to patients, especially in global health. Given the nearly complete lack of diagnostic imaging and superior availability of camera phones in resource poor settings, the use of mobile phones in such settings can substantially improve the accessibility of diagnostic tools, which can be life-saving for the young infants and children most at risk.

Thus, it may be beneficial to provide an exemplary device, apparatus, method and computer-accessible medium for providing and/or facilitating thermal imaging that can be simple to use, and which can overcome at least some of the deficiencies described herein above.

### SUMMARY OF EXEMPLARY EMBODIMENTS

Exemplary of the present disclosure can include an apparatus for generating thermal imaging information of a biological structure(s), including detector arrangement(s) which can be configured to receive and detect light radiation(s), a determination arrangement(s) which can be configured to determine a position or an orientation of the detector arrangement(s) with respect to the biological structure(s) to generate data, and a computer arrangement configured to generate the thermal imaging information based on the radiation and the data when the detector arrangement(s) is moved. The thermal imaging information can include a 2-dimension temperature map(s) of a portion(s) of the biological structure(s). The thermal imaging information can include a 3-dimension temperature map(s) of a portion(s) of the biological structure(s).

In some exemplary embodiments of the present disclosure, an indication of whether there can be an infection or an inflammation in a portion(s) of the biological structure(s) can be provided based on the thermal imaging information. The determination arrangement(s) can be configured to determine at a position(s) or the orientation using a sensor arrangement(s), which can include a gyroscope, an accelerometer or a camera. A plurality of images can be generated based on the radiation prior to the generation of the thermal imaging information. A reference image(s) can be generated based on the plurality of images. The thermal imaging information can be generated based on the reference image(s). It can be determined whether the reference image(s) contains all predetermined elements of the biological structures prior to the generation of the thermal imaging information.

In certain exemplary embodiments of the present disclosure, a diagnosis of the biological structure(s) can be determined based on the thermal imaging information. The diagnosis being determined can include a probability or a likelihood of an infection or an inflammation of the biological structure(s). The diagnosis being determined can include pneumonia. The diagnosis can be based on a temperature difference between a first portion of the biological structure(s) and a different second portion of the biological structure(s).

In another exemplary embodiment can be an exemplary system, method and computer-accessible medium for determining a diagnosis for a biological structure(s), which can include receiving first information related to a plurality of images of the biological structure(s), combining the plurality of images into a reference image(s), where the reference image(s) can include thermal imaging information associated with the biological structure(s), determining a sufficiency of the reference image(s), and determining the diagnosis for the biological structure(s) based on the reference image(s), and as a function of the sufficiency.

In some exemplary embodiments of the present disclosure, the plurality of images can include a thermal image(s) and a visual image(s). The first information can further include of a position or an orientation of the images. The images can be combined into the reference image(s) using a stitching procedure. The sufficiency can include a determination of whether the biological structure(s) can be rotated in the reference image(s) and/or whether the reference image(s) can include all critical elements of the biological structure(s). The diagnosis can include a probability or a likelihood of an infection or an inflammation of the biological structure(s). The diagnosis can include pneumonia.

In a further exemplary embodiment of the present disclosure, an apparatus can be provided for obtaining thermal imaging information of biological structure(s), including a detector arrangement(s) which can be configured to receive and detect a light radiation(s), a controller arrangement(s) which can be configured to control multiple angles or multiple positions at which the light radiation(s) can be received from the biological structure(s), and detected by the detector arrangement(s) to generate data, and a computer arrangement configured to generate the thermal imaging information based on the radiation and the data when the positions or the angles of the receipt of the radiation are changed.

According to still a further exemplary embodiment of the present disclosure, an apparatus can be provided for determining a probability or a likelihood of an infection or an inflammation of biological structure(s), which can include a detector arrangement(s) which can be configured to receive and detect a radiation(s) from the biological structure(s). A computer arrangement can be configured to generate thermal imaging information for a portion(s) of the biological structure(s) based on the radiation, and determine the probability or the likelihood of the infection or the inflammation based on the thermal imaging information. The thermal imaging information can be compared to a further imaging information, and the probability or the likelihood of the infection or the inflammation can be determined based on the comparison. The further imaging information includes a previously-obtained thermal imaging information for the portion(s). The probability or the likelihood of the infection or the inflammation is for pneumonia. The biological sample(s) can be a chest cavity. A recommendation for whether a treatment can be prescribed can be generated based on the probability or the likelihood of the infection or the inflammation.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is an exemplary illustration of exemplary components of an exemplary device/apparatus/system according to an exemplary embodiment of the present disclosure;

FIGS. 2A-2C are exemplary images taken using the exemplary device/apparatus/system according to an exemplary embodiment of the present disclosure shown in FIG. 1;

FIG. 3A is an exemplary image of the exemplary system/device/apparatus according to an exemplary embodiment of the present disclosure;

FIG. 3B is an exemplary screenshot generated using exemplary software programmed on the exemplary system/device/apparatus according to an exemplary embodiment of the present disclosure;

FIG. 3C is an exemplary diagram of the exemplary device/apparatus according to another exemplary embodiment of the present disclosure;

FIG. 4 is an exemplary illustration of a reference chest provided using the exemplary device/apparatus/system, according to an exemplary embodiment of the present disclosure;

FIG. 5 is an exemplary illustration of an acquired infrared image provided using the exemplary device/apparatus/system, according to an exemplary embodiment of the present disclosure;

FIG. 6 is an exemplary illustration of an aligned infrared image provided using the exemplary device/apparatus/system, according to an exemplary embodiment of the present disclosure;

FIG. 7 is an exemplary illustration of an optimally aligned image provided using the exemplary device/apparatus/system, according to an exemplary embodiment of the present disclosure;

FIG. 8 is an exemplary illustration showing temperature elevations at pixel locations provided using the exemplary device/apparatus/system, according to an exemplary embodiment of the present disclosure;

FIG. 9 is a further exemplary illustration showing filtered and post-processed temperature elevations at pixel locations provided using the exemplary device/apparatus/system, according to an exemplary embodiment of the present disclosure;

FIG. 10 is an exemplary flow chart illustrating an exemplary method for generating a thermal image according to an exemplary embodiment of the present disclosure;

FIG. 11 is an exemplary flow chart illustrating an exemplary method for determining a diagnosis for a biological structure according to an exemplary embodiment of the present disclosure; and

FIG. 12 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and appended claims.

### DETAILED DESCRIPTION OF EXEMPLARY EMBODIMENTS

The exemplary system/device/apparatus/method/computer-accessible medium according to exemplary embodiments of the present disclosure can be used to achieve high resolution two-dimensional (“2D”) or three-dimensional (“3D”) temperature maps by utilizing a series of low-resolution thermal images, and can track information obtained from a positional and/or imaging sensor. Such exemplary techniques can facilitate the use of a low-cost thermal sensor or device to create temperature maps that can be greater than the native resolution of the sensor itself, and thus, can be a super-resolution technique. Such exemplary procedures can be important when making a thermal-imaging based medical diagnosis in the resource-poor regions, as this exemplary approach can facilitate the production of clinically useful thermal images without needing and maintaining an expensive high-end thermal camera. Because many low-resolution thermal sensors can be compact and light-weight, this exemplary approach can have the advantage of making portable or embedded thermal measurement systems. This exemplary technique may not be restricted to low-resolution sensors alone. When combined with a high-resolution thermal camera, the exemplary procedures can also produce improved image qualities including of better resolution and signal to noise ratio.

The thermal images can be displayed on devices such as mobile phones, tablets, laptops and other computers. Since health care professionals, and those who care for animals, do not routinely evaluate thermal images, the display device can be coupled with a software application to assist with image interpretation based on an image analysis procedure, and machine based learning of normal and abnormal heat patterns in humans and animals.

Described below is an exemplary system/device/apparatus/method/computer-accessible medium according to exemplary embodiments of the present disclosure that can facilitate a rapid high-resolution thermal imaging using low cost thermal sensors and scanning mechanisms. In an exemplary embodiment, a metallic mirror can reflect not only the visible light, but also an invisible thermal infrared light. One or multiple motorized rotating mirrors (e.g., a Galvo) can be positioned in front of a stationary thermal sensor. By rotating the mirror, or mirrors, e.g., in x/y axes, a spatial thermal image can be acquired. Because a Galvo mirror device can facilitate rapid scanning and precise control of the dwelling time, such method can obtain high resolution and large field-of-view scan within a short time.

According to another exemplary embodiment of the present disclosure, it is possible to use an exemplary multi-pixel, or array, thermal sensor to achieve high resolution scans. An array sensor can be rotated in the x, x/y or x/y/z, /y direction, or any combination thereof. Translation of the sensor can also be performed. The exemplary thermal sensor can be combined with the Galvo mirror(s), as described above. Because an array sensor can have a wide field-of-view, and coarser spatial sampling, a deconvolution method/procedure can be used to interpolate a refined image of high spatial resolution.

The Galvo mirror(s) has/have been conventionally used to scan lasers of visible or infrared range for the purpose of illumination. In an exemplary embodiment, the Galvo mirror(s), with the invisible thermal infrared light at the detection side can be used.

Exemplary embodiments of the exemplary system/device/apparatus can utilize low-cost hardware, thus making it possible to make hand-held high resolution thermal imaging systems coupled with a mobile phone or tablet. Exemplary embodiments can include a low cost mobile phone thermal imager attachment for home insulation and a medical thermal imaging system for fever or inflammatory disease detection.

An exemplary mobile-phone based thermal imaging device according to an exemplary embodiment of the present disclosure can be used to differentiate viral versus bacterial infections in clinical medicine. Clinicians can use this safe, noninvasive, and reliable thermal imaging device as a point of care screening tool to identify patients with likely bacterial infections, especially respiratory infections. This exemplary thermal imaging technique can analyze temperature patterns to predict the probability of bacterial infection. With a simple-to-use thermal camera, the clinician can take a thermal picture of the patient, and receive the interpretation (e.g., computer interpretation) of the likeliness of bacterial infection, facilitating the clinician to make a decision about the need for antibiotic treatment in a single office visit.

The exemplary system/device/apparatus/method/computer-accessible medium according to an exemplary embodiment of the present disclosure can reduce health care costs, improve rational use of antibiotics and minimize radiation exposure to the patient.

In another exemplary embodiment of the present disclosure, an exemplary thermal imaging device can take a thermal picture of the patient, and provide an answer of yes/no for likelihood of bacterial infection. For example, an interpretation of the probability of bacterial infection can be based on correlation with the gold standard of chest x-rays to diagnose pneumonia. According to further exemplary embodiments of the present disclosure, it is possible to provide certain technology to produce a low cost thermal camera facilitating wide spread distribution.

Thermal images can be commonly acquired by thermal cameras. Scientific-grade thermal cameras can be very expensive, currently costing between $5,000 and $50,000. Even a low-grade thermal camera, primarily designed for house insulation inspections (see e.g., Reference 1), can easily cost over $1,000 and may not be feasible for a wide deployment in resource-poor regions. Super low-cost thermal cameras (e.g., less than $100) became possible in recent years due to the emergence of miniature thermal sensors, and low-cost microcontrollers. (See, e.g., Reference 8). As described herein, the exemplary system/device/apparatus/method/computer-accessible medium according to an exemplary embodiment of the present disclosure can be or include a snap-on thermal imaging attachment for mobile phones. This exemplary attachment can contain or include a miniature thermal sensor, a compact and high speed scanning mechanism and a microcontroller within a compact snap-on enclosure that can work with all or most smart phones. A simple and easy to use/operate application (e.g., mobile phone application or app) can be used to acquire thermal images from the attachment and display the results as overlays to the phone's camera view. Advanced 3D thermal map displays can be possible by incorporating exemplary stereo-based surface reconstruction techniques. (See, e.g., Reference 5).

### Exemplary Characteristics

The exemplary system/device/apparatus/method/computer-accessible medium according to an exemplary embodiment of the present disclosure can have the following exemplary characteristics: (i) Instantaneous Use: the device can complete a clinically-usable thermal scan; (ii) Universal Applications: the attachment can be compatible with most smart phones (e.g., Android smartphones, Apple smartphones, Windows smartphones etc.); (iii) Informative Results: the app can facilitate overlaying the thermal images with camera images; and (iv) Easy-to-operate: a healthcare worker with minimal training can perform a quality scan.

### Exemplary Device/System/Apparatus

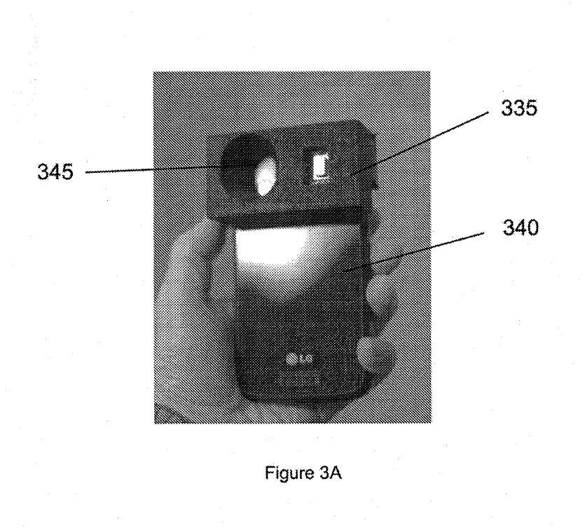

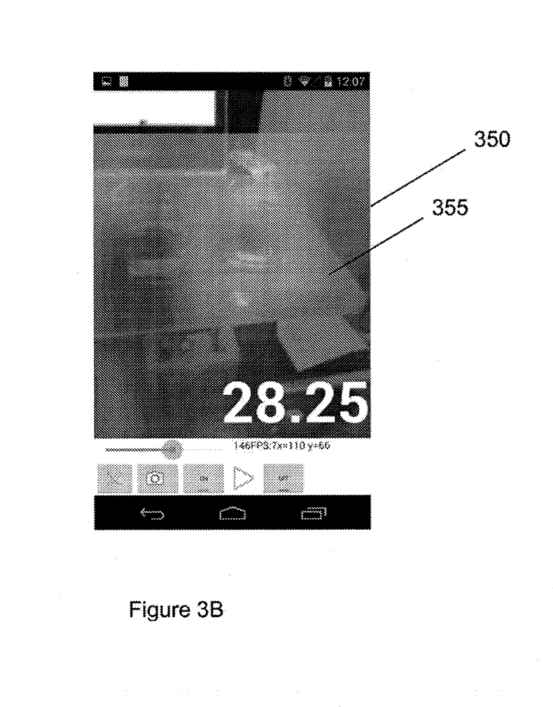

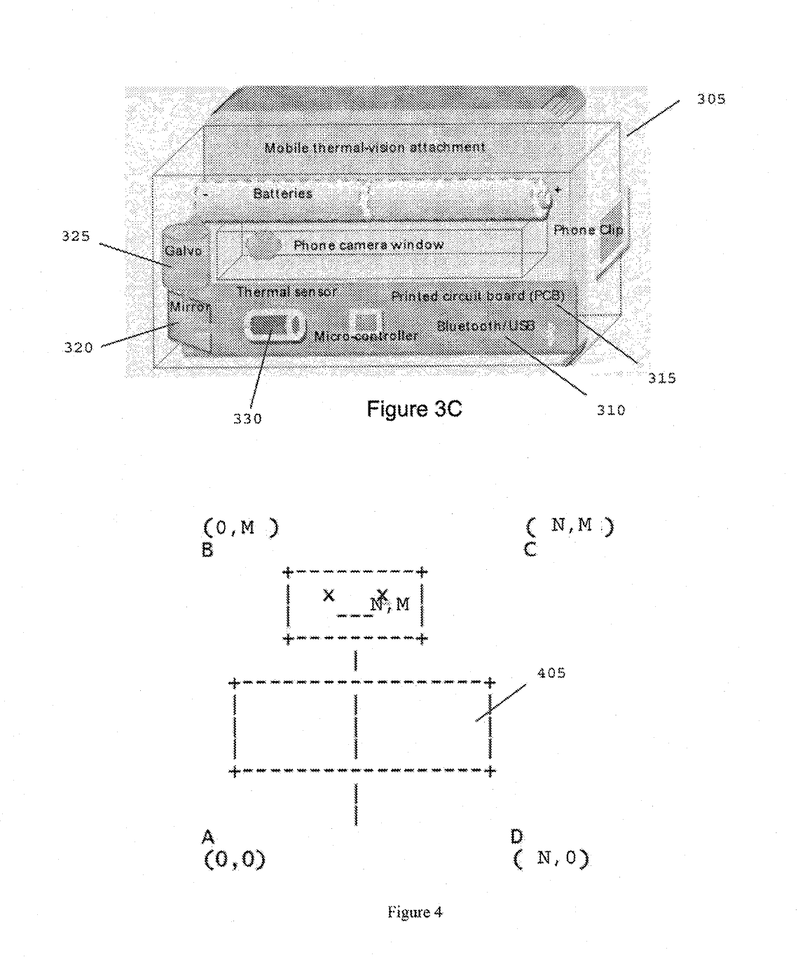

A compact thermal scanner operated with an Android smartphone was developed and has been successfully tested to acquire thermal images from a healthy volunteer. An exemplary illustration of the exemplary thermal scanner attachment is shown in FIG. 1, which can be used with thermal attachment 305 from FIG. 3C. The total cost for the components used was less than $100. A low-cost and versatile microcontroller “IOIO” was used for the initial design. An IOIO can be operated by an Android phone in either through connection medium 310, which can be wired or wireless. Exemplary connection mediums can include a universal serial bus (“USB”) or Bluetooth. The entire exemplary attachment can have a low power consumption, and can be powered by a battery. With the microcontroller IOIO, the entire exemplary system/device/apparatus/method/computer-accessible medium according to an exemplary embodiment of the present disclosure can be integrated into a single printed circuit board 315 (“PCB”) with further reduced cost and much smaller footprint.

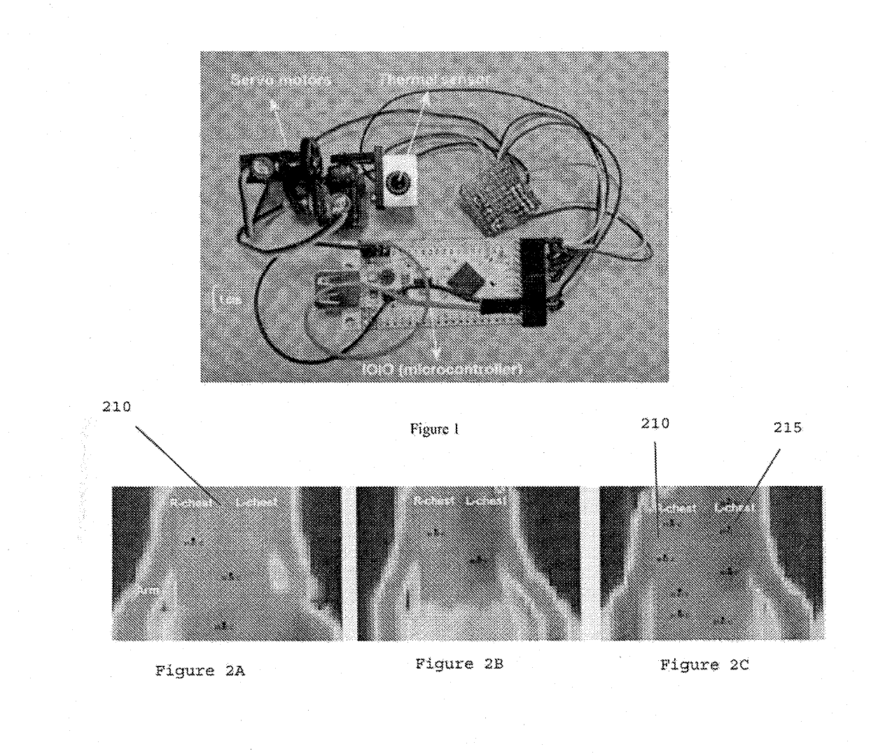

Using the above shown system/device/apparatus/method/computer-accessible medium according to an exemplary embodiment of the present disclosure, thermal images were acquired from a healthy volunteer. FIG. 2A illustrates an exemplary temperature map of the chest area 210 of the subject, sitting about 20 cm from the thermal sensor. The skin temperature ranges from about 31.0° C. to 32.3° C. After the initial scan, the subject was put on a heating pad to the left half of the chest for approximately 1 min. FIG. 2B shows an exemplary image of a temperature distribution immediately after removing the heated pad. Another thermal image, shown in FIG. 2C, was acquired about 2 minutes after taking off the heated pad. From the exemplary image of FIG. 2C, it can be clear that exemplary system/device/apparatus/method/computer-accessible medium according to an exemplary embodiment of the present disclosure can be used to detect a left-to-right chest temperature difference (e.g., temperature difference between the left chest 210 and the right chest 215) of 2° C. or less, which can be sufficient to detect pneumonia reflected by skin temperature differences based on case reports in certain publications. (See, e.g., References 6 and 10).

Scanning speeds to acquire images can be important. For example, each thermal image in FIGS. 2A-2C took about 90 seconds to acquire. This can be sufficient when scanning a child in sleep, however, when imaging throughput can be needed, such as in a busy first-level clinic with many children waiting in line, obtaining accurate images with a slow scanning speed can be difficult. One particular advantage of the exemplary system/device/apparatus can be the reduction of the acquisition time to less than 10 seconds. The speed bottleneck of the current design can be the use of mechanical servo motors. Certain exemplary techniques can be combined to achieve faster speed: (i) the use of a rotating mirror 320 (e.g., Galvo 325) to sweep the field-of-view instead of moving the sensor itself, and (ii) the use of an array of thermal sensors 330 instead of a point sensor. As indicated herein, the Galvo is a rapid scanning device which rotates one or more orthogonal mirrors and, which is commonly used for laser shows/scanners.

The use of the Galvo can obtain more precise scan control and high resolution thermal maps within seconds. Furthermore, there can be low-cost array thermal sensors (e.g., 16×4 and 8×8 pixel arrays) currently available. For the same image size, a 64-pixel array sensor can reduce the acquisition time below 1.5 seconds. The cost and form-factor of an array sensor can be close to a point sensor used in the prototype device; however, the angular resolution of a pixel in an array sensor can be inferior to the use of a point sensor. In order to use an array sensor with the Galvo, exemplary real-time data interpolation and de-convolution scheme(s) can be utilized to achieve both high speed and high resolution. Based on these exemplary techniques, a battery-powered thermal camera attachment shown in FIG. 3C can be provided. For example, the thickness of the attachment can be less than 1 cm. The exemplary mobile phone attachment can also attach using the phone's battery to further reduce cost.

FIG. 3A illustrates an exemplary device/system/apparatus 335 according to an exemplary embodiment of the present disclosure. The exemplary device/system/apparatus 335 can be contained in a plastic enclosure, facilitating its attachment it to attach to a smartphone (e.g., phone 340). A thermal sensor can point towards the target to receive thermal infrared signals. The exemplary device/system/apparatus 335 can be powered by an external battery through a mini-USB port at 5 VDC. The exemplary power source can be an external battery pack for mobile phones, the mobile phone's own battery or a battery holder with standard batteries providing a 5V DC voltage supply. The attachment can have a camera window 345 facilitating the mobile phone camera to take simultaneous images through the attachment enclosure.

The exemplary device/system/apparatus 335 can have dimensions of about 1×2×1 cm, and can capture high speed images (e.g., 7-10 frames per second low-resolution images). The exemplary device/system/apparatus 335 can (i) be Bluetooth enabled, (ii) have low power consumption (e.g., 50 hours of continuous imaging with 3 AA batteries), and (iii) be fully integrated with a smartphone (e.g., an Android, iPhone or Windows Phone smartphone), and can have integrated software with co-registered thermal and visual images.

### Exemplary Software Interface

FIG. 3B is an exemplary screenshot of the exemplary software that programs the exemplary system/device/apparatus can use to execute the exemplary procedures. For example, panel 350 can be graphics user interface (“GUI”) image panel, which can display, in real-time, the images 355 captured from the mobile phone camera. The thermal image overlay, can be as a semi-transparent overlay on top of the camera view. By touching the screen, real-time temperature readings can be obtained (e.g., in Fahrenheit or Celsius), from a given spot. The exemplary software can facilitate a saving of a snapshot of both thermal and visual cameras at any given time. In addition, a “video” of both cameras can be saved, facilitating advanced imaging analysis for super-resolution analysis for the thermal imaging data in the future.

The exemplary software can program the exemplary system/device/apparatus to passively receive the thermal infrared light radiated from a heated body (e.g., a human, living animal, stove or a hot engine). The electromagnetic (“EM”) radiation from this attachment can be limited to the Bluetooth signal used to communicate between the attachment and a smartphone. Because Bluetooth uses very low power EM radiation, it can be very safe for use in human, similar to numerous existing Bluetooth-enabled smartphones, headsets and laptops.

### Exemplary Scanning Procedure

In order to obtain a wide field-of-view high-resolution thermal image in a portable or non-portable device, the following exemplary procedures can be used:

A reference image of the chest 405 can be used. (e.g., see FIG. 4). Points A, B, C and D can represent the edges of the image frame, and can be labeled with their Cartesian coordinates. From this representation, the set of (x, y) coordinates containing lung tissue can be well-known. An infrared thermal sensor can be used to make non-contact measurement of temperature, either at a given point in space, or at a group of locations such as that from an array sensor. A positional sensor can be used to track the position and/or orientation of the thermal sensor. This sensor can be, but is not limited to, a gyroscope, a GPS tracker, an electromagnetic tracker, an optical tracker or a digital camera. The positional information can be coordinated with the thermal images obtained from the thermal sensor, such that for every thermal image measurement, a directional vector and/or a position to the image can be associated.

Thermal images of a given view or region-of-interest can be obtained from at least two positions (e.g., sensor positions) or orientations; this can be done by moving the thermal sensor through either a motorized motion, or an uncontrolled motion, such as a slow hand swipe or shaking. Examples of the uncontrolled motion can include: (i) the shaking produced at a hand-held position, (ii) a slow circular hand-rotation (e.g., a few seconds per cycle), (iii) slow reciprocal hand-twisting (e.g., a few seconds per cycle), (iv) an approximated linear translation and (v) a slow vibrational motion enabled through a spring-loaded sensor enclosure. The acquired infrared image is shown in FIG. 5. The exemplary acquired image can contain at least one thermal reading, and can be more or less pixels, and can be skewed in 3 dimensional space. The symbol “#” (element 505) can indicate an area of inflammation due to pneumonia.

Both thermal images and the positional readings can be obtained from the thermal sensor and the positional sensor, respectively, and the positions/directions of the sensor can be changed (e.g., continuously changed). This can produce a multi-view thermal measurement around the target.

Using the positional information associated with each thermal reading, multi-view low-resolution images can be combined into a high-resolution thermal reading. This can be done by a multi-frame blind deconvolution or an optimization process. For example, a set of coordinates over the target space can be defined. For each position, a temperature reading that best fit, in the least square sense, the thermal measurements of all views can be solved for based on the light-of-sight based on the directional vectors obtained from the positional information.

An exemplary sensor can include a digital camera, and the position and orientation of the camera sensor can be automatically calibrated by an exemplary multi-view stereo (“MSV”) technique. Three dimensional thermal imaging can be facilitated when combining the multi-frame thermal images with the corresponding visual camera images. Applying the exemplary MSV procedure to the visual camera images, a 3D surface of the target and the surrounding structures can be calculated, and a temperature map distribution on this 3D surface can be solved for based on a least-square fitting of the thermal measurements, and the directional information can be restored from the exemplary MSV calculations.

The obtained 2D or 3D thermal images can be displayed on a monitor, facilitating the inspection and manipulation of the images. The scan directions, distance-to-target and the magnitude of the controlled/uncontrolled motion can be adjusted to obtain a refined thermal image that can be adapted based on the interpretation or findings from the initial scan.

In order to conduct the image analysis, the following exemplary procedures can be used. While the procedures below use the patient's chest as an example, it should be noted that the exemplary procedures can be used on any suitable body part or portion of the body.

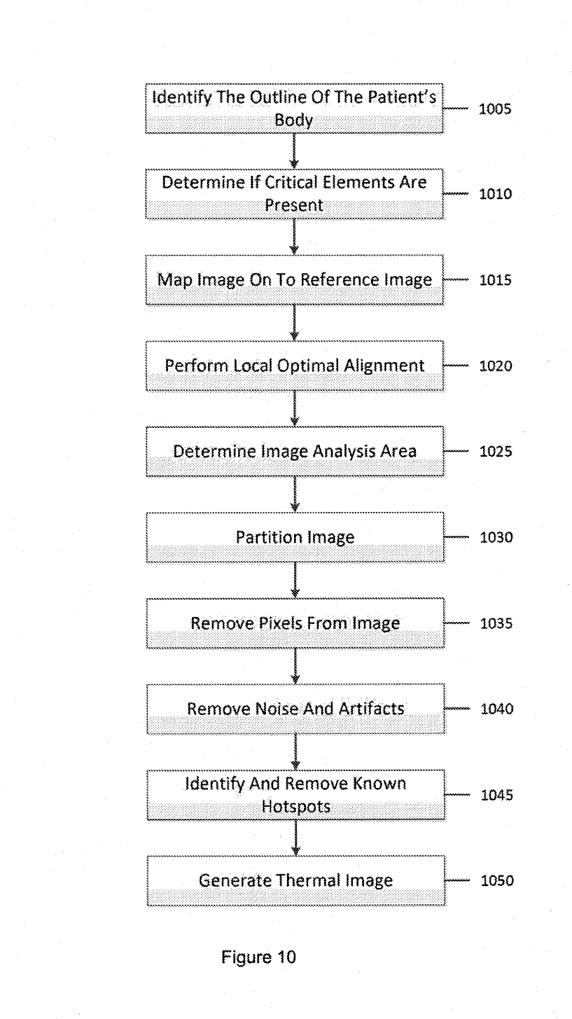

Image registration and morphing can include the processes of mapping one image onto a reference image (e.g., either the exemplary thermal image or the exemplary visual image), while preserving the spatial information present in the original image. The first exemplary procedure for image registration and morphing can be the identification of the outline of the patient's body based on temperature differences between the patient and the external environment (see, e.g., procedure 1005 of FIG. 10). This information can be acquired using exemplary procedure including edge detection, histogramming and segmentation techniques. If this information cannot be obtained from the image, the operator can be informed of the need to acquire additional images. FIG. 5 shows an exemplary acquired image from which the outline 510 of the patient's body can be determined.

If the outline of the patient's body can be determined from the image, then at procedure 1010, using the exemplary system/apparatus/device/method/computer-accessible medium according to an exemplary embodiment of the present disclosure, it can be determined if certain important or critical elements needed for an eventual diagnosis are present. For example, for evaluation of the chest, the shoulders, the axilla and the likely location of the diaphragm should be present. The width of the chest can be determined as the distance between the shoulders and the length of the chest can be at least about 1.5 times the distance between the shoulders extending inferiorly from the level of the shoulders in the exemplary case of a chest. The determination of the sufficiency of the image can be made by locating readily identifiable landmarks in the image, such as the neck and shoulders, and then calculating, based on the size and position within the image of the neck and shoulders, if other key elements of the patient are present. If this information cannot be obtained from the image, the operator can be informed of the need to acquire additional images.

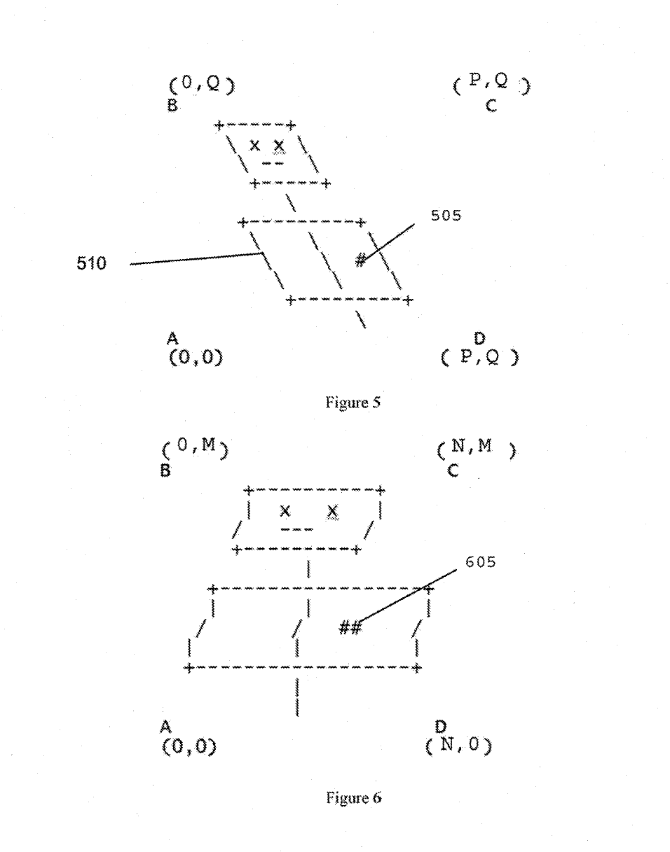

If the image includes the necessary elements of the patient, then, at procedure 1015, the image can be mapped onto a reference image. The mapping procedure can be performed by the application of a series of geometric transformations to the acquired image, which can align the acquired image with the reference image. The transformations can include translation, scaling and rotation around an axis. Exemplary techniques to determine the transformations can include determining the patient's center of mass in the image for translation, determining the patient's size in the image for scaling and determining the patient's skew in the image for rotation. Following these initial transformations, an iterative, hill-climbing technique can be used to find a local optimal alignment (e.g., procedure 1020). The exemplary process can produce a numerical correlation coefficient which can be used to accept or reject the image based on quality of alignment. If the correlation coefficient is not sufficiently high, the operator can be informed of the need to acquire additional images. FIG. 6 shows the exemplary acquired image from FIG. 5 after being aligned to the reference image using a series of geometric transformations.

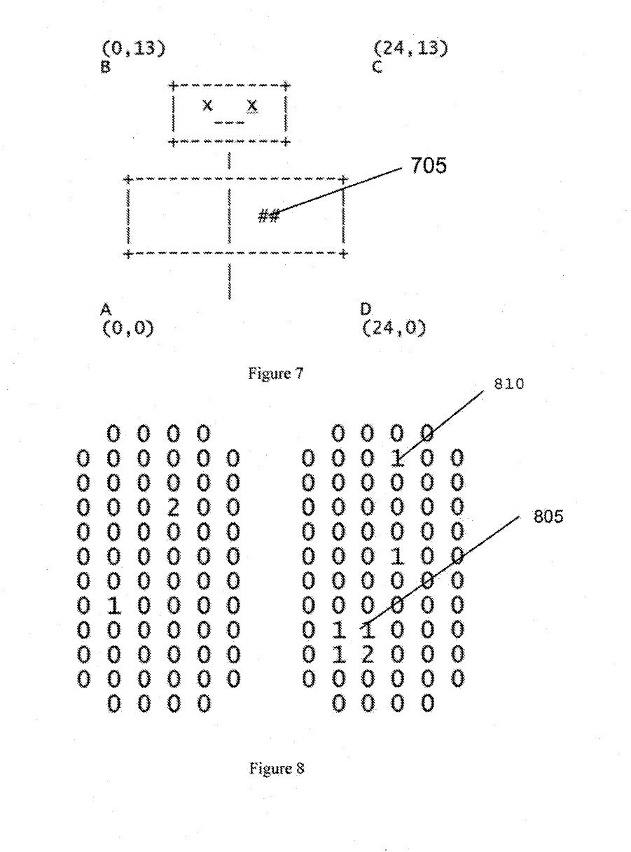

Following the exemplary image registration and morphing procedure above, the acquired infrared image can be optimally aligned to the limits of the geometric transforms employed (e.g. with respect to minimizing sum of squared error) to the reference image. However, the image may not be perfectly aligned. Additional heuristic processing can serve to improve the image alignment even further. Exemplary heuristics can include rules which rely on knowledge of human anatomy, and upon clues that can be derived from the thermal image. Exemplary clues can be typical hot-spots found around the neck and over the xiphoid process. Following the application of these exemplary heuristics, the image can be aligned further as shown in FIG. 7.

When it can be determined that each patient has a sufficient number of images of acceptable quality for further analysis, the exemplary procedure can continue. Alternatively, instead of the all of the images acquired above, one anterior and posterior view can be used. If only one of the anterior or posterior view can be available, this information can be incorporated in the image analysis result. If images are still insufficient, the operator can be informed of the need to acquire additional images.

In order to spatially map the body, with the exemplary device/system/apparatus/method/computer-accessible medium according to an exemplary embodiment of the present disclosure, an exemplary image analysis area can be determined based on a location of the left chest and the right chest and a top and a bottom of each side of the left chest and the right chest (e.g., procedure 1025). Each pixel can have a precise location within each half of the chest. Anterior and posterior images can be aligned for analysis (e.g., image registration). The image can be partitioned (see, e.g., procedure 1030) into areas to be analyzed and areas to be excluded.

Following the exemplary alignment to the reference image, most or all pixels not including lung tissue can be removed from the image (see, e.g., procedure 1035). Known hot areas can be excluded based on the exemplary image library (e.g., axillae, neck and midline spine). Exemplary locations of these hot spots can be based on the initial outline of the image accepted for analysis. FIG. 8 shows the pixel information from just the lung tissue from the aligned image of FIG. 7. A number can be used to indicate for example, the number of 0.1 degree elevations, of temperature relative to the baseline temperature of the lung tissue. The pixel locations of lung tissue can be much fewer, and much more uniform, than the original aligned image from FIG. 7. FIG. 8 shows the region surrounding the area of inflammation (element 805) indicated by the “##’ (element 705) in the acquired image of FIG. 7.

The lung images from FIG. 7 can be further, processed to remove noise and artifact, while maintaining image information necessary to a later diagnosis of the probability of a patient having pneumonia (see e.g., procedure 1040). Exemplary processing can include filtering to eliminate spectral information not consistent with a diagnosis of pneumonia. Additional heuristic processing can include algorithms which can exclude artifacts, such as those introduced by tubes or wires included in the image. Following the application of these exemplary techniques, the region of interest information can be transformed to shown in FIG. 8.

The exemplary processing procedure shown in FIG. 8 can include identifying and characterizing all of the hot-spots in the lungs (see, e.g., procedure 1045). Hot-spots can be characterized using metrics such as size, intensity, shape, regularity, and/or a distribution and location of each hot-spot within the lung. The exemplary measurements can be packaged into a form amenable to processing by the exemplary diagnosis system. This procedure can serve as a bridge between the objective data collection system and the more subjective clinical diagnosis procedure. The packaged data can be applied to the exemplary rules. The exemplary system/device/apparatus/method/computer-accessible medium, according to an exemplary embodiment of the present disclosure, can derive a number of conclusions from the data, which can form the basis of the diagnosis.

Using only the hot-spot information from the previous procedure, the likelihood that “hot” areas can represent pathology can be determined. Areas that can be symmetrically “hot”, and that can be unlikely to be consistent with pathology, can be excluded. The exemplary image library can be used to determine symmetric structures and shapes inconsistent with pathology. Also, hot areas too small in size can be excluded as these can be likely to be due to random variation in temperature. After the hotspots are removed, the exemplary image can be generated (see, e.g., procedure 1050), and the exemplary image library can be used to determine minimum size limits. A probability of the image being consistent with bacterial infection based on asymmetry and/or temperature patterns in right and left chest can be determined. The procedure used to make a diagnosis can also be trainable based on the ever expanding image library. The exemplary conclusions from the exemplary system/device/apparatus/method/computer-accessible medium, according to an exemplary embodiment of the present disclosure, can be consistent with a positive or negative diagnosis of pneumonia. If inconclusive, different rules can be weighted to derive an overall diagnosis along with a confidence measure of the diagnosis. These exemplary weightings can all be determined from training data including, but not limited to, machine learning or exemplary neural network techniques.

In a healthy individual, it can be expected that the skin of the torso over the area of the lungs can be of a uniform temperature. A digital infrared image of a lung uniform in temperature, however, can contain noise and artifacts due to the imperfections of the measurement of infrared energy radiated from the healthy patient. A source of error can be quantization noise due to the conversion of an infinitely variable analog measurement into a discrete digital value. Quantization noise can be characterized as being of high spectral frequency as it can be relatively uncorrelated from one pixel to the next.

The exemplary diagnosis technique/procedure according to an exemplary embodiment of the present disclosure can utilize the assumption that an elevation of skin temperature can be detectable in the skin area adjacent to the area of infection in the lungs. This exemplary elevation in skin temperature can be due to the patient's inflammatory response to the infection. An assumption can also be that the area of inflammation can be large in magnitude relative to the quantization error, and is can be in size relative to the resolution of the detector. This can result in the area of inflammation being of low spectral frequency relative to the frequency of the noise.

FIG. 8 shows an archetypical digital scan of the area adjacent to the two lungs. The average lung temperature has been subtracted from all of the values of the scan, such that only those pixels indicating elevation in temperature above the average are non-zero (e.g., element 805). In this example, it can be assumed that the resolution of the exemplary detector can be 0.1 C. Therefore, a value of 1 (e.g., element 810) shown in FIG. 8 can indicate a temperature of 0.1 degrees above the patient's baseline skin temperature. The area of inflammation is present in FIG. 8, which can be characterized by being large in (i) intensity relative to the resolution of the detector and/or (ii) in extent relative to the size of a single pixel.

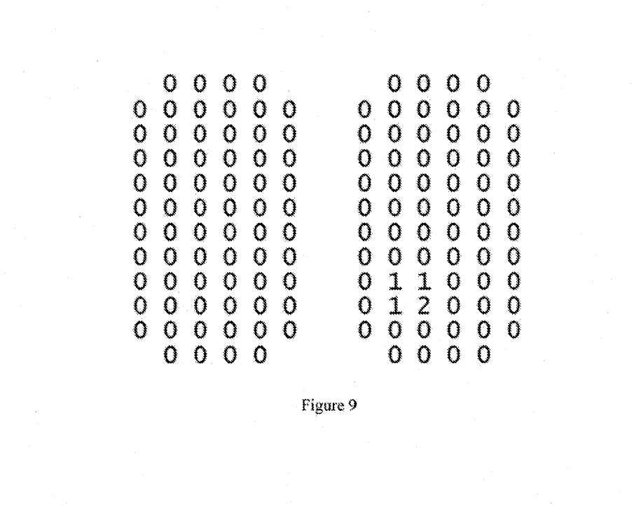

FIG. 8 also provides an illustration indicative of noise in both the left and right lungs. The exemplary noise spikes illustrated in FIG. 8 are shown as being 1 pixel in size, but they may only be small relative to the size of a detectable area of inflammation. The application of 2-dimensional filtering techniques/procedures can be used to eliminate the noise from the image while preserving the characteristics of the area of inflammation. FIG. 9 illustrates the image from FIG. 8 following the application of an exemplary 2-dimensional digital filtering techniques. The parameters of the filters can be derived from the fundamental characteristics of the detector.

The diagnosis of pneumonia from an x-ray, or from an infrared image, may likely only be made by a medical expert. The automated detection system described above applies this knowledge in an exemplary computer program/procedure. This can be achieved through as a rule-based system. Rules within the system can be fed from hot-spot information obtained from the image. Hot spots can be characterized by their size, intensity, shape and location within the lung. Rules can have the form of, for example, “if size>A and intensity>B and shape=elliptical and location=lower-left-lobe, then this can indicate a positive result for pneumonia with a likelihood of D %”. In this example, the values A, B, C, and D can be constants. In the event multiple rules apply to a set of observations, other rules can exist to form an overall diagnosis from the other rules that “fired”.

As described herein, the values of A, B, C and D can be determined from training images. An expert can diagnoses a number of training images as being positive or negative for pneumonia. Well-known optimization techniques can then be used to automatically adjust all constants of the exemplary system/method/computer-accessible medium such that the diagnosis generated from the exemplary system/method/computer-accessible medium for the training images can closely match the diagnosis made by the expert. The assumption can be that if the training set can be large enough, then the expert and the exemplary system/method/computer-accessible medium can derive the same diagnosis for new images not part of the original set of training images. The rules and parameters can be adjusted as new training images become available.

In addition to the above, multiple thermal sensors can be used simultaneously to achieve faster image acquisition. There can be a positional sensor associated with each thermal sensor or a single positional sensor for the entire thermal sensor group as long as the relative positions between the thermal sensors can be known. The 2D and 3D thermal maps can be acquired continuously so that the temperature variations over time at any given position can be restored. This can make it possible for real-time monitoring of temperature of the target. If the thermal sensor has a higher temporal resolution than the positional sensor readings, an interpolation between the adjacent positional readings can be assumed to correspond to the intermediate thermal image frames. A fiducial marker can also be used, which can be visible in both the thermal images and the digital camera images to make an automatic correspondence between the two sets of measurements.

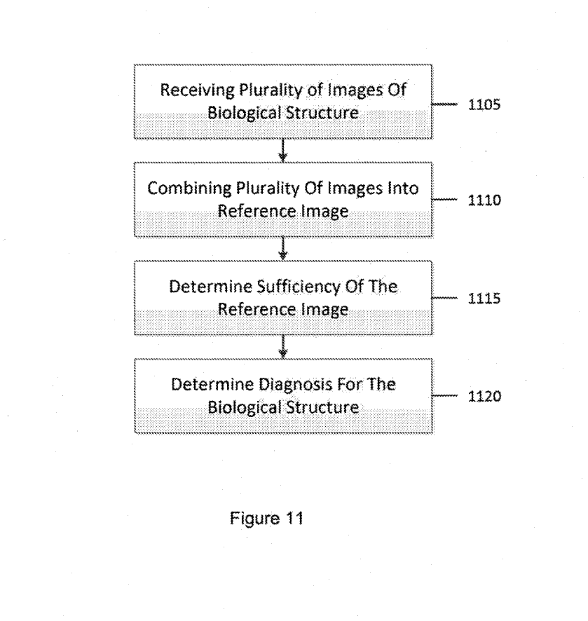

FIG. 11 is an exemplary flow chart illustrating an exemplary method of determining a diagnosis for a biological structure according to an exemplary embodiment of the present disclosure. For example, at procedure 1105, a plurality of images of the biological structure can be received. At procedure 1110, the plurality of images can be combined into one or more reference images (e.g., using an exemplary stitching procedure). The sufficiency of the one or more reference images can be determined at 1115, which can include determining if all of the critical elements needed for a diagnosis are present and/or determining if the biological structure is rotated in the reference images. At procedure 1120, the diagnosis for the biological structure can be determined (e.g., using the exemplary methods described above).

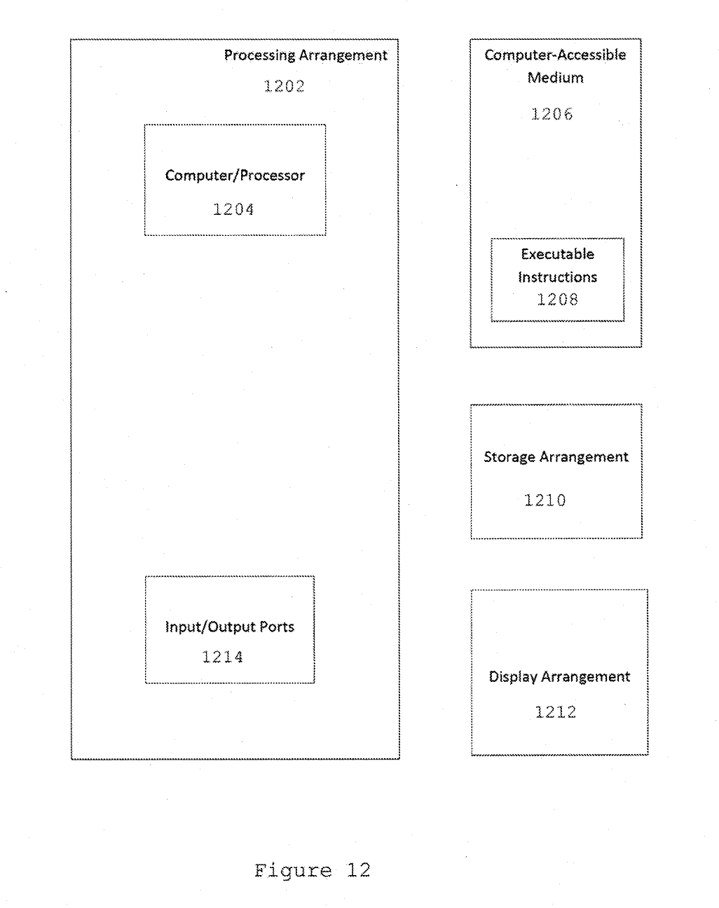

FIG. 12 shows a block diagram of an exemplary embodiment of a system according to the present disclosure. For example, exemplary procedures in accordance with the present disclosure described herein can be performed by a processing arrangement and/or a computing arrangement 1202. Such processing/computing arrangement 1202 can be, for example entirely or a part of, or include, but not limited to, a computer/processor 1204 that can include, for example one or more microprocessors, and use instructions stored on a computer-accessible medium (e.g., RAM, ROM, hard drive, or other storage device).

As shown in FIG. 12, for example a computer-accessible medium 1206 (e.g., as described herein above, a storage device such as a hard disk, floppy disk, memory stick, CD-ROM, RAM, ROM, etc., or a collection thereof) can be provided (e.g., in communication with the processing arrangement 1202). The computer-accessible medium 1206 can contain executable instructions 1208 thereon. In addition or alternatively, a storage arrangement 1210 can be provided separately from the computer-accessible medium 1206, which can provide the instructions to the processing arrangement 1202 so as to configure the processing arrangement to execute certain exemplary procedures, processes and methods, as described herein above, for example.

Further, the exemplary processing arrangement 1202 can be provided with or include an input/output arrangement 1214, which can include, for example a wired network, a wireless network, the internet, an intranet, a data collection probe, a sensor, etc. As shown in FIG. 12, the exemplary processing arrangement 1202 can be in communication with an exemplary display arrangement 1212, which, according to certain exemplary embodiments of the present disclosure, can be a touch-screen configured for inputting information to the processing arrangement in addition to outputting information from the processing arrangement, for example. Further, the exemplary display 1212 and/or a storage arrangement 1210 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entirety.
[1] Allen2004—Allen T L, Lee D N, Chan K J, Snow G L. (2004) Feasibility and utility of infrared thermography to identify acute appendicitis, Annals of Emergency Medicine, 44(4): S31
[2] Duke2001—Duke T, Mgone J, Frank D. (2001) Hypoxaemia in children with severe pneumonia in Papua New Guinea. Int. J. Tuberc. Lung Dis. 5:511-519. PMID: 11409576
[3] eMarketer2013—“Mobile/SmartPhones,” January 2013, URL: http://www.newmediatrendwatch.com/world-overview/98-mobile-devices.
[4] Fieldston2013—Fieldston E, Erwiesch, C, Altschuler, S. (2013) Application of business model innovation to enhance value in health care delivery, JAMA Pediatrics, 167(5):409-411.
[5] Funikawa2010—Furukawa Y, Ponce J (2010) Accurate, dense, and robust multiview stereopsis. Pattern Analysis and Machine Intelligence, IEEE Transactions. 1362-1376.
[5] Onyango 1993—Onyango F E, Steinhoff M C, Wafula E M, Wariva S, Musia J, Kitonyi J. (1993) Hypoxaemia in young Kenyan children with acute lower respiratory infection. BMJ. 306:612-615. PMID: 8369033
[6] Potanin1970—Potanin C. (1970) Thermographic patterns of pulmonary disease, Chest, 58(5): 491-6.
[7] Scott2008—Scott J A, Brooks W A, Pe iris JS, Holtzman D, Mu !holland E K. (2008) Pneumonia research to reduce childhood mortality in the developing world. J Clin Invest., 118(4): 1291-1300.
[8] Schneider2012—Schneider D, “IR Eye—a cheap infrared scanner can replace an expensive thermal-imaging camera”, IEEE Spectrum Magazine
[9] Smyth1998—Smyth A, Carty H, Hart C A. (1998) Clinical predictors of hypoxaemia in children with pneumonia. Ann. Trop. Paediatr. 18:31-40. PMID: 9691999
[10] Wiecek2001—Wiecek B, Peszynski-Drews C, Wysocki M, Jakubowska T, Danych R, Zwolenik S. (2001) Advanced thermal image processing for medical and biological applications. Eng Med and Bioi Soc, Proc. of the 23rd Annual International Conference of the IEEE, 3: 2805-2807.
[11] UNICEF2012—Committing to Child Survival: A Promise Renewed-Progress report, New York, 2012. URL: http://www.apromiserenewed.org

1. An apparatus for generating thermal imaging information of at least one biological structure, comprising:
at least one detector arrangement which is configured to receive and detect at least one light radiation;
at least one determination arrangement which is configured to determine at least one of a position or an orientation of the at least one detector arrangement with respect to the at least one biological structure to generate data; and
a computer arrangement configured to generate the thermal imaging information based on the radiation and the data when the at least one detector arrangement is moved.

2. The apparatus according to claim 1, wherein the thermal imaging information includes at least one multi-dimension temperature map of at least one portion of the at least one biological structure.

3. (canceled)

4. The apparatus according to claim 1, wherein the computer arrangement is further configured to provide an indication of whether there is an infection or an inflammation in at least one portion of the at least one biological structure based on the thermal imaging information.

5. The apparatus of claim 1, wherein the at least one determination arrangement is configured to determine at least one of the position or the orientation using at least one sensor arrangement.

6. The apparatus of claim 1, wherein the at least one sensor arrangement includes at least one of a gyroscope, an accelerometer or a camera.

7. The apparatus of claim 1, wherein the computer arrangement is further configured to generate a plurality of images based on the radiation prior to the generation of the thermal imaging information.

8. The apparatus of claim 7, wherein the computer arrangement is further configured to generate at least one reference image based on the plurality of images.

9. The apparatus of claim 8, wherein the thermal imaging information is generated based on the at least one reference image.

10. The apparatus of claim 8, wherein the computer arrangement is further configured to determine if the at least one reference image contains all predetermined elements of the biological structures prior to the generation of the thermal imaging information.

11. The apparatus of claim 1, wherein the computer arrangement is further configured to determine a diagnosis of the at least one biological structure based on the thermal imaging information.

12. The apparatus of claim 11, wherein the diagnosis being determined includes at least one of (i) a probability or a likelihood of at least one of an infection or an inflammation of the at least one biological structure, or (ii) pneumonia.

13. (canceled)

14. The apparatus of claim 11, wherein the computer arrangement is further configured to determine the diagnosis based on a temperature difference between a first portion of the at least one biological structure and a different second portion of the at least one biological structure.

15-32. (canceled)

33. A system for determining a diagnosis for at least one biological structure, comprising:
a computer hardware arrangement configured to:
receive first information related to a plurality of images of the at least one biological structure;
combine the plurality of images into at least one reference image, wherein the at least one reference image includes thermal imaging information associated with the at least one biological structure;
determine a sufficiency of the at least one reference image; and
determine the diagnosis for the at least one biological structure based on the at least one reference image, and as a function of the sufficiency.

34. The system of claim 33, wherein the plurality of images include at least one thermal image and at least one visual image.

35. The system of claim 33, wherein the first information further includes at least one of a position or an orientation of the plurality of images.

36. The system of claim 33, wherein the plurality of images are combined into the at least one reference image using a stitching procedure.

37. The system of claim 33, wherein the sufficiency includes a determination of at least one of (i) whether the at least one biological structure is rotated in the at least one reference image, or (ii) if the at least one reference image includes all critical elements of the at least one biological structure.

38. (canceled)

39. The system of claim 33, wherein the diagnosis includes at least one of (i) a probability or a likelihood of at least one of an infection or an inflammation of the at least one biological structure (ii) pneumonia.

40. (canceled)

41. The system of claim 33, wherein the computer hardware arrangement is further configured to determine the diagnosis based on a temperature difference between a first portion of the at least one biological structure and a different second portion of the at least one biological structure.

42. An apparatus for obtaining thermal imaging information of at least one biological structure, comprising:
at least one detector arrangement which is configured to receive and detect at least one light radiation;
at least one controller arrangement which is configured to control multiple angles or multiple positions at which the at least one light radiation is received from the at least one biological structure, and detected by the at least one detector arrangement to generate data; and
a computer arrangement configured to generate the thermal imaging information based on the radiation and the data when the positions or the angles of the receipt of the radiation are changed.

43-53. (canceled)

54. An apparatus for determining a probability or a likelihood of an infection or an inflammation of at least one biological structure, comprising:
at least one detector arrangement which is configured to receive and detect at least one radiation from the at least one biological structure; and
a computer arrangement configured to:
generate thermal imaging information for at least one portion of the at least one biological structure based on the radiation, and
determine the probability or the likelihood of the infection or the inflammation based on the thermal imaging information.

55-67. (canceled)
